(19) United States
(12) Patent Application Publication
(10) Pub. No.: US 2024/0410609 A1
(43) Pub. Date: Dec. 12, 2024

(54) AIR CONDITIONING DEVICE, AIR SENSOR DEVICE, AND CONTROL METHOD THEREOF

(71) Applicant: Samsung Electronics Co., Ltd., Suwon-si (KR)

(72) Inventors: Jun HWANG, Suwon-si (KR); Seokkyun KIM, Suwon-si (KR); Taewoo KIM, Suwon-si (KR); Kilsoo SON, Suwon-si (KR); Sangwoo LEE, Suwon-si (KR); Ohkun CHUNG, Suwon-si (KR)

(21) Appl. No.: 18/811,580

(22) Filed: Aug. 21, 2024

Related U.S. Application Data
(63) Continuation of application No. PCT/KR2023/002423, filed on Feb. 21, 2023.

(30) Foreign Application Priority Data
Mar. 28, 2022 (KR) 10-2022-0038221

(51) Int. Cl. F24F 11/64; F24F 11/65; F24F 110/10

(52) U.S. Cl. CPC F24F 11/64; F24F 11/65; F24F 2110/10

(57) ABSTRACT

Disclosed is an air conditioning device. The air conditioning device comprises: a communication interface including a circuit; a temperature sensor; a compressor; and at least one processor, comprising processing circuitry, individually and/or collectively, configured to control the operation of the compressor based on a set temperature value set in the air conditioning device, a first temperature value acquired by the temperature sensor, and a second temperature value received from the air sensor device through the communication interface. At least one processor, individually and/or collectively, may be configured to: identify the difference between the first temperature value and the second temperature value, and, based on the identified difference being at least a threshold value, control the operation of the compressor based on the set temperature value and the first temperature value.

### CROSS-REFERENCE TO RELATED APPLICATIONS

This application is a continuation of International Application No. PCT/KR2023/002423 designating the United States, filed on Feb. 21, 2023, in the Korean Intellectual Property Receiving Office and claiming priority to Korean Patent Application No. 10-2022-0038221, filed on Mar. 28, 2022, in the Korean Intellectual Property Office, the disclosures of each of which are incorporated by reference herein in their entireties.

### BACKGROUND

### Field

The disclosure relates to an air conditioning device, an air sensor device and a control method thereof, and for example, to an air conditioning device that operates performing inter-communication, an air sensor device, and a control method thereof.

### Description of Related Art

Recently, air conditioning devices for maintaining a comfortable indoor environment by adjusting temperature, humidity, cleanliness, airflow, and the like of an indoor space are being supplied.

Because air conditioning devices of the related art operate taking into consideration a current indoor temperature and a setting temperature, there has been a disadvantage of not being able to operate taking into consideration a sensory temperature of a user, a surrounding temperature at which the user is positioned, or the like.

At times, there has been a disadvantage of causing displeasure to the user due to excessive cooling or excessive heating by continuously performing cooling or heating functions despite the sensory temperature of the user having reached a desired temperature of the user (or, the setting temperature of the user).

A portable air sensor device and the like may sense the surrounding temperature in which the user is positioned by being positioned adjacently to the user, and has an advantage of appropriately performing the cooling or heating function if the air conditioning device operates using a temperature value sensed by the air sensor device.

However, the above-described advantage is manifested when the air sensor device is positioned adjacently to the user, or when positioned so as to appropriately sense the surrounding temperature at which the user is positioned, and has a problem of the disadvantage of causing displeasure to the user due to excessive cooling or excessive heating by continuously performing cooling or heating functions despite the sensory temperature of the user having reached the desired temperature of the user (or, the setting temperature of the user) still occurring if the air sensor device is not positioned adjacently to the user.

Accordingly, there has been a continuous demand for an air conditioning device that predicts (or, identifies) a position of the air sensor device, and appropriately uses the temperature value sensed by the air sensor device and a control method thereof.

### SUMMARY

Embodiments of the disclosure provide an air conditioning device which operates in connection with an air sensor device, and operates by appropriately using a temperature value sensed by the air sensor device, the air sensor device, and a control method thereof.

According to an example embodiment, an air conditioning device includes: a communication interface including circuitry, a temperature sensor, a compressor, and at least one processor, comprising processing circuitry, individually and/or collectively, configured to: control an operation of the compressor based on a setting temperature value set from the air conditioning device, a first temperature value obtained by the temperature sensor, and a second temperature value received from an air sensor device through the communication interface; identify a difference value between the first temperature value and the second temperature value; and control, based on the identified difference value being greater than or equal to a threshold value, an operation of the compressor based on the setting temperature value and the first temperature value.

At least one processor, individually and/or collectively, may be configured to: identify, based on the identified difference value being greater than or equal to a threshold value, a relationship between a position at which air discharged from the air conditioning device reaches and a current position of the air sensor device, and transmit information that guides of a position change of the air sensor device based on the identified relationship to the air sensor device through the communication interface.

At least one processor, individually and/or collectively, may be configured to: identify, based on the identified difference value being greater than or equal to a threshold value, that the air sensor device is disposed at a position at which air discharged from the air conditioning device directly reaches, and transmit information that guides of the position change of the air sensor device to the air sensor device through the communication interface.

At least one processor, individually and/or collectively, may be configured to: identify, based on the air conditioning device operating in a cooling mode, whether a difference value of subtracting the second temperature value from the first temperature value is greater than or equal to a first threshold value, and operate, based on the identified difference value being greater than or equal to the first threshold value, the compressor for a cooling function to be performed based on the setting temperature and the first temperature value.

At least one processor, individually and/or collectively, may be configured to operate, based on the difference value being less than the first threshold value, the compressor for the cooling function to be performed based on the setting temperature and the second temperature value.

At least one processor, individually and/or collectively, may be configured to: identify, based on the air conditioning device operating in a heating mode, whether a difference value of subtracting the first temperature value from the second temperature value is greater than or equal to a second threshold value, and operate, based on the identified difference value being greater than or equal to the second threshold value, the compressor for a heating function to be performed based on the setting temperature and the first temperature value.

At least one processor, individually and/or collectively, may be configured to: operate, based on the difference value being less than the second threshold value, the compressor for the heating function to be performed based on the setting temperature and the second temperature value.

At least one processor, individually and/or collectively, may be configured to: set a third threshold value as the threshold value based on the first temperature value and the second temperature value being obtained at a time-point at which a first time is passed from an operation starting time-point of the air conditioning device, and set a fourth threshold value different from the third threshold value as the threshold value based on the first temperature value and the second temperature value being obtained at a time-point at which a second time different from the first time is passed from an operation starting time-point of the air conditioning device.

At least one processor, individually and/or collectively, may be configured to set the fourth threshold value to the third threshold value to a relatively small value based on the second time being relatively longer than the first time.

At least one processor, individually and/or collectively, may be configured to: transmit the first temperature value to an external server through the communication interface, and control, based on a control signal for controlling an operation of the compressor being received from the external server according to the difference value of the first temperature value and the second temperature value, an operation of the compressor based on the control signal.

According to an example embodiment, a method of controlling an air conditioning device including a temperature sensor includes: obtaining a first temperature value through the temperature sensor, and controlling an operation of a compressor of the air conditioning device based on a setting temperature value set from the air conditioning device, the first temperature value, and a second temperature value received from an air sensor device, and the controlling includes identifying a difference value between the first temperature value and the second temperature value, and controlling, based on the identified difference value being greater than or equal to a threshold value, an operation of the compressor based on the setting temperature value and the first temperature value.

The method may further include: identifying, based on the identified difference value being greater than or equal to the threshold value, a relationship between a position at which air discharged from the air conditioning device reaches and a current position of the air sensor device, and transmitting information that guides of a position change of the air sensor device to the air sensor device based on the identified relationship.

The identifying may include: identifying, based on the identified difference value being greater than or equal to the threshold value, that the air sensor device is disposed at a position at which air discharged from the air conditioning device directly reaches.

The controlling may include: identifying, based on the air conditioning device operating in a cooling mode, whether a difference value of subtracting the second temperature value from the first temperature value is greater than or equal to a first threshold value, and operating, based on the identified difference value being greater than or equal to the first threshold value, the compressor for a cooling function to be performed based on the setting temperature and the first temperature value.

The controlling may include: operating, based on the difference value being less than the first threshold value, the compressor for the cooling function to be performed based on the setting temperature and the second temperature value.

The controlling may include: identifying, based on the air conditioning device operating in a heating mode, whether a difference value of subtracting the first temperature value from the second temperature value is greater than or equal to a second threshold value, and operating, based on the identified difference value being greater than or equal to the second threshold value, the compressor for a heating function to be performed based on the setting temperature and the first temperature value.

The controlling may include: operating, based on the difference value being less than the second threshold value, the compressor for the heating function to be performed based on the setting temperature and the second temperature value.

The method may further include: setting a third threshold value as the threshold value based on the first temperature value and the second temperature value being obtained at a time-point at which a first time is passed from an operation starting time-point of the air conditioning device, and setting a fourth threshold value different from the third threshold value as the threshold value based on the first temperature value and the second temperature value being obtained at a time-point at which a second time different from the first time is passed from an operation starting time-point of the air conditioning device.

The setting a fourth threshold value may include setting the fourth threshold value to the third threshold value to a relatively small value based on the second time being relatively longer than the first time.

The method may further include: transmitting the first temperature value to an external server, and the controlling may include controlling, based on a control signal for controlling an operation of the compressor being received from the external server according to a difference value of the first temperature value and the second temperature value, an operation of the compressor based on the control signal.

According to various example embodiments of the disclosure, based on the air sensor device being determined as appropriately sensing the surrounding temperature of the user, the air conditioning device may be operated using the temperature value sensed by the air sensor device, and based on the air sensor device being determined as not appropriately sensing the surrounding temperature of the user, the air conditioning device may be operated using the temperature value sensed by the air conditioning device.

Accordingly, the air conditioning device may operate taking into consideration a sensory temperature of the user, and an indoor temperature in which the user is positioned.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a diagram illustrating an air conditioning device and an air sensor device according to various embodiments;

FIG. 2 is a block diagram illustrating an example configuration of an air conditioning device according to various embodiments;

FIG. 3 is a block diagram illustrating an example configuration of an air sensor device according to various embodiments;

FIG. 4 is a diagram illustrating communication between an air conditioning device and an air sensor device according to various embodiments;

FIG. 5 is a diagram illustrating a difference between a first temperature value and a second temperature value according to various embodiments;

FIG. 6 is a diagram illustrating guide information according to various embodiments;

FIG. 7 is a diagram illustrating an incorrect position of an air sensor device according to various embodiments;

FIG. 8 is a diagram illustrating a heating mode according to various embodiments;

FIG. 9 is a flowchart illustrating an example method of controlling an air conditioning device in a cooling mode according to various embodiments;

FIG. 10 is a flowchart illustrating an example method of controlling an air conditioning device in a heating mode according to various embodiments; and

FIG. 11 is a signal flow diagram illustrating example communication between an air conditioning device and an air sensor device according to various embodiments.

### DETAILED DESCRIPTION

Terms used in the disclosure will be briefly described, and the disclosure will be described in greater detail.

The terms used in an embodiment of the disclosure are general terms selected that are currently widely used considering their function herein. However, the terms may change depending on intention, legal or technical interpretation, emergence of new technologies, and the like of those skilled in the related art. Further, in certain cases, there may be terms arbitrarily selected, and in this case, the meaning of the term will be disclosed in greater detail in the relevant description. Accordingly, the terms used herein are not to be understood simply as its designation but based on the meaning of the term and the overall context of the disclosure.

Various modifications may be made to the embodiments of the disclosure, and there may be various types of embodiments. Accordingly, specific embodiments will be illustrated in drawings, and described in detail in the detailed description. However, it should be noted that the various embodiments are not for limiting the scope of the disclosure to a specific embodiment, but they should be interpreted to include all modifications, equivalents or alternatives of the embodiments included in the ideas and the technical scopes disclosed herein. In case it is determined that in describing the embodiments, detailed description of related known technologies may unnecessarily confuse the gist of the disclosure, the detailed description will be omitted.

Terms such as “first,” and “second” may be used in describing the various elements, but the elements are not to be limited by the terms. The terms may be used only to distinguish one element from another.

A singular expression includes a plural expression, unless otherwise clearly specified in context. It is to be understood that the terms such as “form” or “include” are used herein to designate a presence of a characteristic, number, step, operation, element, component, or a combination thereof, and not to preclude a presence or a possibility of adding one or more of other characteristics, numbers, steps, operations, elements, components or a combination thereof.

The term “module” or “part” used in the disclosure perform at least one function or operation, and may be implemented with a hardware or software, or implemented with a combination of hardware and software. In addition, a plurality of “modules” or a plurality of “parts,” except for a “module” or a “part” which needs to be implemented as a specific hardware, may be integrated in at least one module and implemented as at least one processor (not shown).

Embodiments of the disclosure will be described in greater detail below with reference to the accompanying drawings to aid in the understanding of those of ordinary skill in the art. However, the disclosure may be implemented in various different forms and is not limited to the embodiments described herein. Further, in the drawings, parts not relevant to the description may be omitted, and like reference numerals may be used for like parts throughout the whole of the disclosure to clearly describe the disclosure.

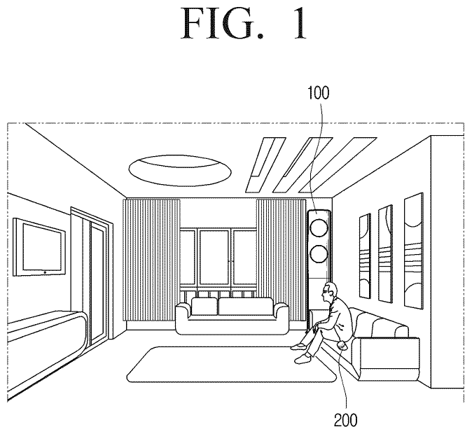

FIG. 1 is a diagram illustrating an example air conditioning device and an air sensor device according to various embodiments.

An air conditioning device 100 according to an embodiment of the disclosure may refer to devices of various types which comfortably maintain indoors through heating, cooling, decreasing humidity (or, dehumidification), humidifying, ventilation, and the like as a conditioning device, an air adjusting device, or a conditioning system.

In an example, the air conditioning device 100 may be implemented as a hot air fan, a heater, an air-conditioner capable of both cooling and heating, and the like. However, the air conditioning device 100 is not limited to the above, and may be implemented in devices of various types which can increase and decrease indoor temperature, and the disclosure may be applied to even devices that can only operate any one from among cooling or heating. For convenience of description below, the air conditioning device 100 will be described as an air-conditioner capable of both cooling and heating.

The air conditioning device 100 according to an embodiment of the disclosure may include an indoor unit and an outdoor unit. The indoor unit may be connected with the outdoor unit, and the indoor unit may exchange refrigerant with the outdoor unit through a pipe. The air conditioning device 100 formed with the indoor unit and the outdoor unit may include various operation modes such as, cooling which lowers an indoor air temperature, heating which raises the indoor air temperature, wind blowing which forms an air flow indoors, dehumidification which lowers indoor humidity, and the like. The operation modes of the air conditioning device 100 will be described below.

The outdoor unit according to an embodiment of the disclosure may exchange heat with external air. The outdoor unit may exchange heat with external air through a cooling cycle that emits heat transferred through the refrigerant from the indoor unit to the outside, and exchanges heat with the external air through a heating cycle that absorbs heat lost by the refrigerant from the outside. The outdoor unit may include a compressor for compressing the refrigerant. Then, the compressor may be implemented into any one from among a constant speed type, a step type (or TPS), and an inverter type. The constant speed type may be a form that controls on and off of driving of the compressor according to a cooling and heating load. The step type may include a plurality of compressors, and may be a form that controls a number of compressors that drives according to the cooling and heating load. The inverter type may be a form that controls increasing and decreasing a driving capability of the compressor linearly according to the cooling and heating load.

The air conditioning device 100 according to an embodiment of the disclosure may perform communication with an air sensor device 200.

The air sensor device 200 may refer, for example, to a separate device distinguished from the air conditioning device 100 and configured to monitor the temperature, air quality, and the like of indoor air.

For example, the air sensor device 200 may sense the temperature, humidity, fine dust concentration (e.g., PM 10, PM 2.5, and PM 1 values, etc.), and the like of surrounding air of the air sensor device 200.

The air sensor device 200 may be referred to as an air monitoring device, and the like, but will be collectively referred to as the air sensor device 200 for convenience of description below, and the air sensor device 200 shown in FIG. 1 is merely one example for convenience of description, and is not limited thereto. For example, the air sensor device 200 may also be implemented as a portable user terminal device that includes a sensor which can sense the temperature, air quality, and the like of the surrounding air.

The air sensor device 200 according to an embodiment of the disclosure may be implemented to be portable, and may sense a surrounding air temperature and a surrounding air quality of a user by being positioned adjacently to the user positioned indoors (e.g., an occupant).

For example, the air sensor device 200 may transmit the sensed surrounding air temperature, surrounding air quality, and the like to the air conditioning device 100, and the air conditioning device 100 may perform an operation based on the surrounding air temperature and the surrounding air quality received from the air sensor device 200.

The air conditioning device 100 according to an embodiment of the disclosure may control an operation of the air conditioning device 100 using the surrounding air temperature of the air sensor device 200 received from the air sensor device 200 rather than controlling an operation of the air conditioning device 100 (e.g., on and off of the compressor, etc.) using only the sensor (e.g., a temperature sensor) provided in the air conditioning device 100.

If the air sensor device 200 is positioned adjacently to the user, because the air conditioning device 100 operates using the surrounding air temperature of the user sensed through the air sensor device 200 in addition to the temperature sensed on its own, air of a more appropriate strength and temperature may be discharged in a surrounding environment of the user (e.g., the surrounding air temperature of the user).

In another example, if the air sensor device 200 is disposed somewhat spaced apart from the user, the surrounding air temperature sensed by the air sensor device 200 may be somewhat different from the surrounding air temperature of the user. In still another example, if the air sensor device 200 is disposed at a position at which air discharged from the air conditioning device 100 directly reaches, an air temperature sensed by the air sensor device 200 may be somewhat different from the surrounding air temperature of the user, or the indoor air temperature (hereinafter, an indoor temperature) in which the user is positioned.

In this case, if the air conditioning device 100 operates using the surrounding air temperature received from the air sensor device 200, a problem may occur of the air conditioning device 100 discharging air of an inappropriate strength and temperature (e.g., air of an excessive cooling or excessive heating temperature) in the surrounding environment of the user (e.g., the surrounding air temperature of the user).

According to various embodiments of the disclosure, a method for determining whether the air conditioning device 100 is disposed at a position at which the air sensor device 200 can appropriately sense the surrounding air temperature of the user using the surrounding air temperature received from the air sensor device 200 or a position at which the temperature of indoor air in which the user is positioned can be appropriately sensed will be described below.

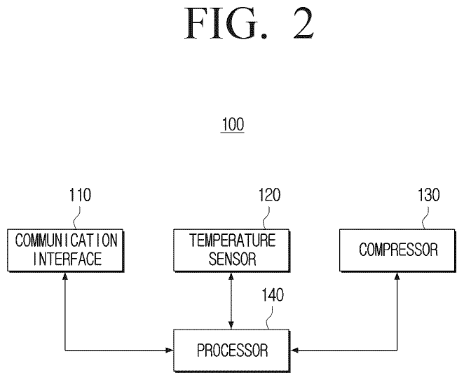

FIG. 2 is a block diagram illustrating an example configuration of an air conditioning device according to various embodiments.

The air conditioning device 100 according to an embodiment may include a communication interface (e.g., including communication circuitry) 110, a temperature sensor 120, a compressor 130, and a processor (e.g., including processing circuitry) 140.

The communication interface 110 according to an embodiment of the disclosure may include various communication circuitry and receive input of data of various types and information by performing communication with external devices. For example, the communication interface 110 may receive input of data of various types, information, and the like from home appliances (e.g., display device, air-conditioner, air purifier, etc.) external storage mediums (e.g., a USB memory), external servers (e.g., WEB HARD), and the like through communication methods such as, for example, and without limitation, an AP-based Wi-Fi (e.g., Wi-Fi, wireless LAN network), Bluetooth, ZigBee, a wired/wireless local area network (LAN), a wide area network (WAN), Ethernet, IEEE 1394, a high-definition multimedia interface (HDMI), a universal serial bus (USB), a mobile high-definition link (MHL), Audio Engineering Society/European Broadcasting Union (AES/EBU), Optical, Coaxial, or the like.

For example, the communication interface 110 according to an embodiment may perform communication with the air sensor device 200, and receive the temperature sensed by the air sensor device 200 from the air sensor device 200, for example, the surrounding air temperature of the air sensor device 200 (hereinafter, referred to as a second temperature value). However, the disclosure is not limited thereto, and may receive air quality sensed by the air sensor device 200 from the air sensor device 200, for example, humidity, fine dust concentration, and the like of the surrounding air of the air sensor device 200.

The air conditioning device 100 according to an embodiment of the disclosure may sense the temperature of the surrounding air (hereinafter, a first temperature value) of the air conditioning device 100 through the temperature sensor 120.

The temperature of the surrounding air (e.g., the first temperature value) of the air conditioning device 100 sensed by the temperature sensor 120 may refer, for example, to the indoor temperature in which the air conditioning device 100 is disposed. For example, the temperature sensor 120 may obtain the first temperature value by sensing the temperature of air (e.g., indoor air) suctioned through the indoor unit of the air conditioning device 100.

The air conditioning device 100 according to an embodiment of the disclosure may further include, in addition to the temperature sensor 120, a wind speed sensor which senses an indoor wind speed, a humidity sensor which senses the humidity in air, a fine dust sensor which senses the fine dust concentration, an olfactory sensor which senses smell, and the like, and may sense a surrounding air quality of the air conditioning device 100 using the sensors of various types.

The air conditioning device 100 according to an embodiment of the disclosure may include the compressor 130. If the compressor 130 is operated according to control of the processor 140, the air conditioning device 100 may discharge heat transferred through the refrigerant to the outside when the air conditioning device 100 is in a cooling mode (e.g., decreasing indoor temperature), and the air conditioning device 100 may absorb heat lost by the refrigerant from the outside (e.g., increasing indoor temperature) when the air conditioning device 100 is in a heating mode.

According to an embodiment of the disclosure, the processor 140 may include various processing circuitry and may, for example, be implemented as a digital signal processor (DSP) that processes a digital signal, a microprocessor, or a time controller (TCON). However, the disclosure is not limited thereto, and may include, for example, and without limitation, one or more from among a central processing unit (CPU), a micro controller unit (MCU), a micro processing unit (MPU), a controller, an application processor (AP), a communication processor (CP), an ARM processor, or an artificial intelligence (AI) processor, or may be defined by the relevant term. In addition, the processor 140 may be implemented with a System on Chip (SoC) or a large scale integration (LSI) in which a processing algorithm is embedded, and may be implemented in a form of a field programmable gate array (FPGA). The processor 140 may perform various functions by executing computer executable instructions stored in a memory 130. The processor 140 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

For example, the processor 140 may control an operation of the compressor 130 based on a setting temperature value set from the air conditioning device 100, the first temperature value obtained (or sensed) by the temperature sensor 120, and the second temperature value received from the air sensor device 200 through the communication interface 110.

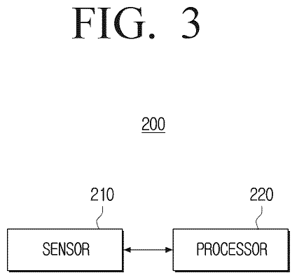

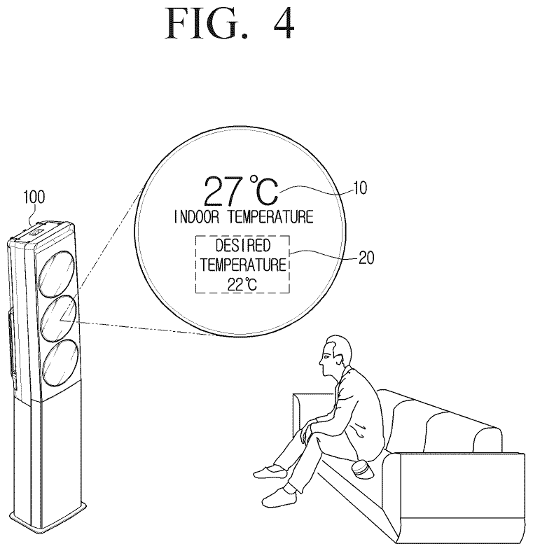

FIG. 3 is a block diagram illustrating an example configuration of an air sensor device according to various embodiments. A more detailed description thereof will be described below with reference to FIG. 4.

The air sensor device 200 according to an embodiment of the disclosure may include a sensor 210 and a processor (e.g., including processing circuitry) 220.

The sensor 210 according to an embodiment may sense the surrounding air temperature (e.g., the second temperature value) of the air sensor device 200. In an example, if the air sensor device 200 is positioned adjacently to the user, the second temperature value sensed by the sensor 210 may correspond to the surrounding air temperature of the user.

The sensor 210 may include a temperature sensor, and may further include, in addition to the temperature sensor, the wind speed sensor which senses the indoor wind speed, the humidity sensor which senses the humidity in air, the fine dust sensor which senses the fine dust concentration, the olfactory sensor which senses smell, and the like to sense the surrounding air quality of the air sensor device 200.

The processor 220 according to an embodiment of the disclosure may be implemented as the digital signal processor (DSP) that processes a digital signal, the microprocessor, or the time controller (TCON). However, the disclosure is not limited thereto, and may include, for example, and without limitation, one or more from among the central processing unit (CPU), the micro controller unit (MCU), the micro processing unit (MPU), the controller, the application processor (AP), the communication processor (CP), the ARM processor, or the artificial intelligence (AI) processor, or may be defined by the relevant term. In addition, the processor 220 may be implemented with the System on Chip (SoC) or the large scale integration (LSI) in which a processing algorithm is embedded, and may be implemented in the form of the field programmable gate array (FPGA). The processor 220 may perform various functions by executing computer executable instructions stored in the memory 130. The processor 220 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

For example, the processor 220 may transmit the second temperature value sensed by the sensor 210, the surrounding air quality of the air sensor device 200, or the like to the air conditioning device 100 through a communication interface (not shown) provided in the air sensor device 200.

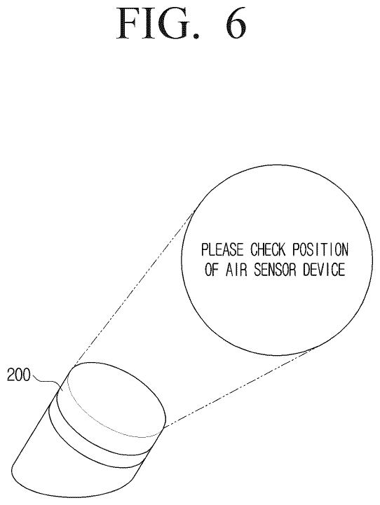

The processor 220 according to an embodiment may provide, based on a notification, guide information, or the like being received from the air conditioning device 100, provide the same. Detailed descriptions thereof will be described below with reference to FIG. 6.

FIG. 4 is a diagram illustrating example communication between an air conditioning device and an air sensor device according to various embodiments.

For example, the processor 140 may control an operation of the compressor 130 based on a setting temperature value 20 set from the air conditioning device 100, a first temperature value 10 obtained (or, sensed) by the temperature sensor 120, the second temperature value received from the air sensor device 200 through the communication interface 110.

The setting temperature value 20 may refer, for example, to an indoor temperature to be reached by operating the air conditioning device 100, and may be referred to as a desired temperature. For example, the air conditioning device 100 may operate the compressor 130 until the first temperature value 10 obtained by the temperature sensor 120 reaches the setting temperature value 20 corresponding to a user setting.

According to an embodiment of the disclosure, the air conditioning device 100 may operate the compressor 130 until the second temperature value obtained by the air sensor device 200 reaches the setting temperature value 20 in addition to the first temperature value 10 obtained by the temperature sensor 120.

For example, the air sensor device 200 may be positioned adjacently to the user, and in this case, the second temperature value obtained by the air sensor device 200 may correspond to the surrounding air temperature of the user. Accordingly, the air conditioning device 100 may control an operation of the compressor 130 (e.g., may adjust strength and temperature of air discharged by the air conditioning device 100) for the surrounding air temperature of the user to reach the setting temperature value 20 (or, until the surrounding air temperature of the user reaches the setting temperature value 20).

In another example, if the air sensor device 200 is not positioned adjacently to the user, the second temperature value obtained by the air sensor device 200 may be different from the surrounding air temperature of the user. In still another example, if the air sensor device 200 is disposed at a position at which air discharged from the air conditioning device 100 directly reaches, the second temperature value obtained by the air sensor device 200 may be different from the indoor temperature. Even in this case, if the air conditioning device 100 operates using the surrounding air temperature received from the air sensor device 200, a problem of the air conditioning device 100 discharging air of an inappropriate strength and temperature (e.g., air of excessive cooling or excessive heating) to the surrounding environment of the user (e.g., the surrounding air temperature of the user) may occur.

According to an embodiment, a method in which the air conditioning device 100 determines, using the second temperature value received from the air sensor device 200, whether the air sensor device 200 is disposed at a position at which the surrounding air temperature of the user can be appropriately sensed or, at a position at which the indoor air temperature in which the user is positioned can be appropriately sensed will be described in greater detail below.

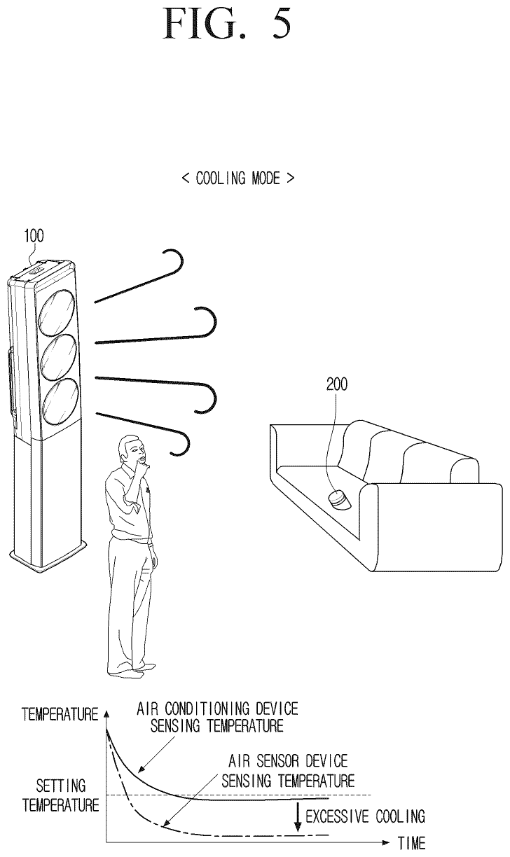

FIG. 5 is a diagram illustrating an example difference between a first temperature value and a second temperature value according to various embodiments.

Referring to FIG. 5, the air conditioning device 100 according to an embodiment of the disclosure may operate in the cooling mode.

The processor 140 according to an embodiment may identify, based on the second temperature value being received from the air sensor device 200, a difference value between the first temperature value 10 (e.g., the temperature value obtained by the temperature sensor 120 provided in the air conditioning device 100) and the second temperature value.

The processor 140 may control, based on the difference value being greater than or equal to a threshold value, an operation of the compressor 130 based on the first temperature value 10 and the setting temperature value 20.

The processor 140 according to an embodiment may identify, based on the difference between the first temperature value 10 and the second temperature value being greater than or equal to a threshold value, a relationship between the position at which air discharged from the air conditioning device 100 reaches and a current position of the air sensor device 200.

For example, if the air conditioning device 100 operates in the cooling mode, it may be assumed that the air sensor device 200 is disposed at a position at which air discharged from the air conditioning device 100 directly reaches.

In this case, the second temperature value obtained by the air sensor device 200 which is disposed at a position at which air discharged from the air conditioning device 100 directly reaches may differ from the first temperature value 10 obtained by the air conditioning device 100 by a threshold value or more prior to the air discharged from the air conditioning device 100 being diffused indoors, or the indoor temperature decreasing.

For example, a time-point prior to the air discharged from the air conditioning device 100 being diffused indoors, or prior to the indoor temperature decreasing may refer, for example, to a point in time at which a first time is passed from an operation starting time-point of the air conditioning device 100.

The processor 140 according to an embodiment may identify a difference value between the first temperature value 10 and the second temperature value at a time-point at which the first time is passed from the operation starting time-point of the air conditioning device 100. Here, the first time may be between 3 minutes to 5 minutes, but this is merely one example, and may be variously changed.

The processor 140 may identify, based on the difference value between the first temperature value 10 and the second temperature value being greater than or equal to a threshold value, that the air sensor device 200 is disposed at a position at which air discharged from the air conditioning device 100 directly reaches.

$${Tr}_{air - conditioningdevice} - {Tr}_{airsensordevice} \geq 5 ° C . \qquad [ Equation 1 ]$$

The Tr_{air-conditioningdevice} may be the first temperature value, Tr_{airsensordevice} may be the second temperature value, and 5° C. may be an example of the threshold value when in the cooling mode.

The processor 140 may identify, based on Equation 1 being satisfied, that air sensor device 200 is disposed at a position at which air discharged from the air conditioning device 100 directly reaches, and that the air sensor device 200 is not disposed at a position at which the surrounding air temperature of the user can be appropriately sensed or, at a position at which the indoor air temperature in which the user is positioned can be appropriately sensed (e.g., identify as the air sensor device 200 being disposed at an incorrect position). The processor 140 may operate the compressor 130 using the first temperature value 10 and the setting temperature value 20.

As shown in FIG. 5, if the air conditioning device 100 is in the cooling mode, the air conditioning device 100 may discharge relatively cool air. According to an embodiment, if the air sensor device 200 is disposed at a position at which the cool air directly reaches, the second temperature value obtained by the air sensor device 200 may be relatively lower than the first temperature value 10 obtained by the air conditioning device 100.

In this case, according to a method of the related art, there is concern that the processor 140 may turn-off, based on determining that the indoor temperature has reached the setting temperature value 20 based on the second temperature value received from the air sensor device 200, the compressor 130 by erroneously determination despite the indoor temperature not having reached the setting temperature value 20. Accordingly, the processor 140 according to an embodiment of the disclosure may operate, based on the difference value between the first temperature value 10 and the second temperature value being greater than or equal to a threshold value, the compressor 130 for a cooling function to be performed using the first temperature value 10 and the setting temperature value 20.

In another example, the processor 140 may identify, based on the difference value between the first temperature value 10 and the second temperature value being less than a threshold value, that the air sensor device 200 is disposed at a position at which the surrounding air temperature of the user can be appropriately sensed or, at a position the indoor air temperature in which the user is positioned can be appropriately sensed. Then, the processor 140 may operate the compressor 130 for the cooling function to be performed using the second temperature value and the setting temperature value 20.

For example, the processor 140 may maintain an operation of the compressor 130 based on the difference value between the first temperature value 10 and the second temperature value being less than a threshold value, and the second temperature value not having reached the setting temperature value 20. In another example, the processor 140 may stop the operation of the compressor 130 based on the second temperature value reaching the setting temperature value 20.

The processor 140 according to an embodiment of the disclosure may identify, based on the difference value between the first temperature value 10 and the second temperature value being greater than or equal to a threshold value (e.g., the first temperature value 10 being greater than the second temperature value by the threshold value or more), that the air sensor device 200 is disposed at a position at which air discharged from the air conditioning device 100 directly reaches, and transmit information guiding of a position change of the air sensor device 200 to the air sensor device 200 through the communication interface 110.

FIG. 6 is a diagram illustrating example guide information according to various embodiments.

Referring to FIG. 6, the air sensor device 200 may display information guiding of the position change.

For example, the processor 140 of the air conditioning device 100 may identify, based on the difference value between the first temperature value 10 and the second temperature value being greater than or equal to a threshold value, that the air sensor device 200 is disposed at a position at which air discharged from the air conditioning device 100 directly reaches.

The air conditioning device 100 may transmit guide information which shows a position change of the air sensor device 200 being required (e.g., ‘please check position of air sensor device’) to the air sensor device 200 for the air sensor device 200 to be disposed at a position at which the surrounding air temperature of the user can be appropriately sensed or, at a position at which the indoor air temperature in which the user is positioned can be appropriately sensed.

The air sensor device 200 may display the guide information. In another example, the air sensor device 200 may also output a sound corresponding to the guide information (e.g., a sound corresponding to ‘please check the position of the air sensor device’) through a speaker (not shown).

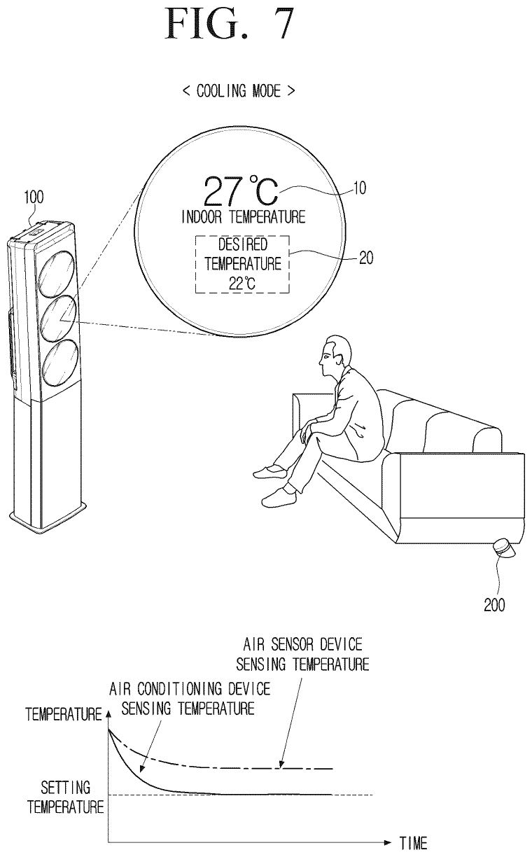

FIG. 7 is a diagram illustrating an example of an incorrect position of an air sensor device according to various embodiments.

FIG. 7 shows the air sensor device 200 not being disposed at a position at which the indoor air temperature in which the user is positioned can be appropriately sensed such as, in addition to a position at which air discharged from the air conditioning device 100 directly reaches, the air sensor device being disposed indoors different from the air conditioning device 100, or under furniture disposed at another indoors.

The processor 140 according to an embodiment may identify, based on the second temperature value being received from the air sensor device 200, the difference value between the first temperature value 10 (e.g., the temperature value obtained by the temperature sensor 120 provided in the air conditioning device 100) and the second temperature value.

According to an embodiment, the second temperature value obtained by the air sensor device 200 which is disposed at a position at which air discharged from the air conditioning device 100 is not able to reach (e.g., indoors different from the air conditioning device 100) may differ from the first temperature value 10 obtained by the air conditioning device 100 by a threshold value or more even after the air discharged from the air conditioning device 100 has been sufficiently diffused or the indoor temperature has decreased.

As illustrated in FIG. 7, if the air conditioning device 100 is in the cooling mode, the air conditioning device 100 may discharge cool air. According to an embodiment, if the air sensor device 200 is disposed at a position at which air discharged from the air conditioning device 100 it not able to reach, the second temperature value obtained by the air sensor device 200 may be relatively higher than the first temperature value 10 obtained by the air conditioning device 100 (e.g., a temperature value higher than the first temperature value 10 by a threshold value or more).

In this case, there is concern that the processor 140 may continuously turn-on, based on determining that the indoor temperature has not reached the setting temperature value 20 based on the second temperature value received from the air sensor device 200, the compressor 130 by erroneously determination despite the indoor temperature having reached the setting temperature value 20. Accordingly, the processor 140 according to an embodiment of the disclosure may turn-off, based on the difference value between the first temperature value 10 and the second temperature value being greater than or equal to the threshold value (e.g., the second temperature value being greater than the first temperature value 10 by the threshold value or more), the compressor 130 using the first temperature value 10 and the setting temperature value 20.

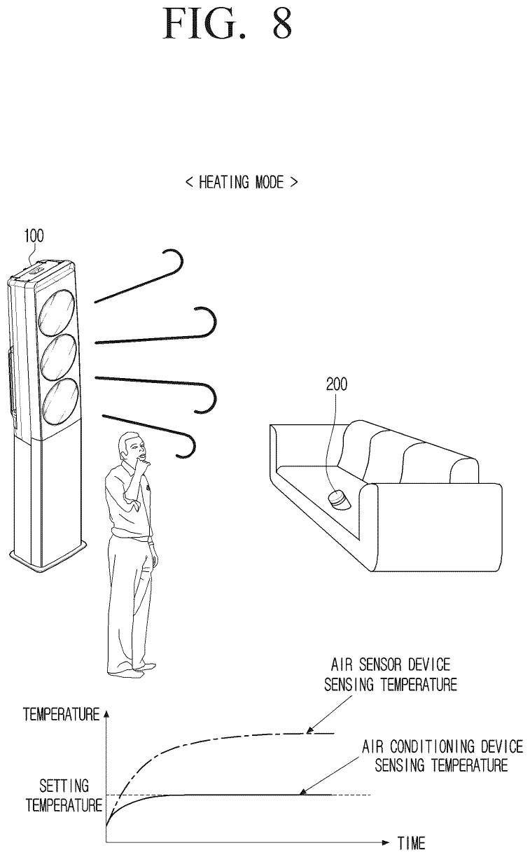

FIG. 8 is a diagram illustrating an example heating mode according to various embodiments.

According to an embodiment, it may be assumed that the air conditioning device 100 is operating in the heating mode, and that the air sensor device 200 is disposed at a position at which air discharged from the air conditioning device 100 directly reaches.

In this case, the second temperature value obtained by the air sensor device 200 which is disposed at a position at which air discharged from the air conditioning device 100 directly reaches may differ from the first temperature value 10 obtained by the air conditioning device 100 by a threshold value or more prior to the air discharged from the air conditioning device 100 being diffused indoors, or the indoor temperature decreasing.

The processor 140 according to an embodiment of the disclosure may identify, based on the difference value between the first temperature value 10 and the second temperature value being greater than or equal to the threshold value, that the air sensor device 200 is disposed at a position at which air discharged from the air conditioning device 100 directly reaches.

$${Tr}_{airsensordevice} - {Tr}_{air - conditioningdevice} \geq 7 ° C . \qquad [ Equation 2 ]$$

The Tr_{air-conditioningdevice} may be the first temperature value, the Tr_{airsensordevice} may be the second temperature value, and 7° C. is an example of the threshold value when in the heating mode. The threshold value for when in the cooling mode and the threshold value for when in the heating mode may vary.

The processor 140 may identify, based on Equation 2 being satisfied, that air sensor device 200 is disposed at a position at which air discharged from the air conditioning device 100 directly reaches, and that the air sensor device 200 is not disposed at a position at which the surrounding air temperature of the user can be appropriately sensed or, at a position at which the indoor air temperature in which the user is positioned can be appropriately sensed (e.g., identify as the air sensor device 200 being disposed at an incorrect position). Then, the processor 140 may operate the compressor 130 using the first temperature value 10 and the setting temperature value 20.

As illustrated in FIG. 8, if the air conditioning device 100 is in the heating mode, the air conditioning device 100 may discharge relatively warm air. According to an embodiment, if the air sensor device 200 is disposed at a position at which the warm air directly reaches, the second temperature value obtained by the air sensor device 200 may be relatively higher than the first temperature value 10 obtained by the air conditioning device 100.

In this case, according to a method of the related art, there is concern that the processor 140 may turn-off, based on determining that the indoor temperature has reached the setting temperature value 20 based on the second temperature value received from the air sensor device 200, the compressor 130 by erroneously determination despite the indoor temperature not having reached the setting temperature value 20. Accordingly, the processor 140 according to an embodiment of the disclosure may operate, based on the difference value between the first temperature value 10 and the second temperature value being greater than or equal to the threshold value, the compressor 130 for a heating function to be performed using the first temperature value 10 and the setting temperature value 20.

In another example, the processor 140 may identify, based on the difference value between the first temperature value 10 and the second temperature value being less than the threshold value, that the air sensor device 200 is disposed at a position at which the surrounding air temperature of the user can be appropriately sensed or, at a position at which the indoor air temperature in which the user is positioned can be appropriately sensed. Then, the processor 140 may operate the compressor 130 for the heating function to be performed using the second temperature value and the setting temperature value 20.

For example, the processor 140 may maintain, based on the difference value between the first temperature value 10 and the second temperature value being less than the threshold value, the operation of the compressor 130 if the second temperature value has not reached the setting temperature value 20. In another example, the processor 140 may stop, based on the second temperature value having reached the setting temperature 20, an operation of the compressor 130.

The processor 140 according to an embodiment of the disclosure may identify, based on the difference value between the first temperature value 10 and the second temperature value being greater than or equal to the threshold value (e.g., the second temperature value being greater than the first temperature value 10 by the threshold value or more), that the air sensor device 200 is disposed at a position at which air discharged from the air conditioning device 100 directly reaches, and transmit information guiding of the position change of the air sensor device 200 to the air sensor device 200 through the communication interface 110.

In still another example, if the air conditioning device 100 operates in the heating mode as shown in FIG. 7, the processor 140 may determine, based on the difference value between the first temperature value 10 and the second temperature value being greater than or equal to the threshold value (e.g., the first temperature value 10 being greater than the second temperature value by the threshold value or more), that the air sensor device 200 is not disposed at a position at which the indoor air temperature in which the user is positioned can be appropriately sensed such as an indoors different from the air conditioning device 100, or under a furniture disposed in another indoors, or outdoors. Then, the processor 140 may operate the compressor 130 for the heating function to be performed using the first temperature value 10 and the setting temperature value 20.

Referring back to FIG. 5, the processor 140 according to an embodiment of the disclosure may set, based on the first temperature value 10 and the second temperature value being obtained at a time-point at which the first time (e.g., 3 minutes to 5 minutes) is passed from the operation starting time-point of the air conditioning device 100, a first threshold value as the threshold value.

In addition, the processor 140 may set, based on the first temperature value 10 and the second temperature value being obtained at a time-point at which the second time (e.g., 10 minutes) is passed from the operation starting time-point of the air conditioning device 100, a second threshold value as the threshold value.

For example, if the air conditioning device 100 operates in the cooling mode, the processor 140 may identify, in the first time, whether the difference value between the first temperature value 10 and the second temperature value is greater than or equal to the first threshold value (e.g., 5° C.) based on Equation 1.

In another example, if the air conditioning device 100 operates in the cooling mode, the processor 140 may identify, in the second time, whether the difference value between the first temperature value 10 and the second temperature value is greater than or equal to the second threshold value (e.g., 3° C.) based on Equation 3.

$${Tr}_{airsensordevice} - {Tr}_{air - conditioningdevice} \geq 3 ° C . \qquad [ Equation 3 ]$$

The Tr_{air-conditioningdevice} may be the first temperature value, the Tr_{airsensordevice} may be the second temperature value, and 3° C. may be an example of the second threshold value when in the cooling mode.

According to an embodiment, the threshold value corresponding to the second time (e.g., the second threshold value) may be a value relatively lower than the threshold value corresponding to the first time (e.g., the first threshold value).

Referring back to FIG. 8, in another example, if the air conditioning device 100 operates in the heating mode, the processor 140 may identify, in the first time, whether the difference value between the first temperature value 10 and the second temperature value is greater than or equal to the first threshold value (e.g., 7° C.) based on Equation 2.

In another example, if the air conditioning device 100 operates in the heating mode, the processor 140 may identify, in the second time, whether the difference value between the first temperature value 10 and the second temperature value is greater than or equal to the second threshold value (e.g., 7° C.) based on Equation 4.

$${Tr}_{air - conditioningdevice} - {Tr}_{airsensordevice} \geq 5 ° C . \qquad [ Equation 4 ]$$

The Tr_{air-conditioningdevice} may be the first temperature value, the Tr_{airsensordevice} may be the second temperature value, and 5° C. may be an example of the second threshold value when in the heating mode.

According to an embodiment, the threshold value corresponding to the second time (e.g., the second threshold value) may be a value relatively smaller than the threshold value corresponding to the first time (e.g., the first threshold value).

The first time and the second time may be variously changed according to a manufacturer of the air conditioning device 100, a user setting, time at which the air sensor device 200 transmits the second temperature value, or the like. In another example, the first time and the second time may be changed according to the setting temperature value 20, the first temperature value 10, the second temperature value, and the like. For example, if a difference between the setting temperature value 20 and the first temperature value 10 is relatively great from the operation starting time-point of the air conditioning device 100, the processor 140 may set the first time to a time longer than 3 minutes to 5 minutes (e.g., 7 minutes), and set the second time to after a certain time (e.g., 5 minutes) from the first time. However, this is merely one example, and is not limited thereto.

The various embodiments of the disclosure may be performed by the processor 140 provided in the air conditioning device 100, but is not limited thereto, and may be performed by the processor 220 provided in the air sensor device 200.

For example, the air sensor device 200 may transmit a signal controlling an operation of the compressor to the air conditioning device 100 after identifying whether the difference between the first temperature value 10 and the second temperature value is greater than or equal to the threshold value,

In still another example, the air conditioning device 100 may transmit the first temperature value 10 to an external server, and the air sensor device 200 may transmit the second temperature value to the external server. Then, the external server may transmit, after identifying whether the difference between the first temperature value and the second temperature value is greater than or equal to the threshold value, a signal controlling an operation of the compressor to the air conditioning device 100.

According to various embodiments of the disclosure, the air conditioning device 100 may identify the difference value between the first temperature value 10 and the second temperature value, and identify whether the identified difference value is greater than or equal to the threshold value. Then, the air conditioning device 100 may control an operation of the air conditioning device 100 using the first temperature value 10 without taking into consideration the second temperature value received from the air sensor device 200 according to the identified result. However, the above is merely one example, and is not limited thereto.

For example, the air conditioning device 100 may identify a difference value between a first humidity (e.g., humidity sensed by a sensor provided in the air conditioning device 100) and a second humidity (e.g., humidity received from the air sensor device 200), and identify whether the identified difference value is greater than or equal to a threshold value. Then, the air conditioning device 100 may control an operation of the air conditioning device 100 using the first humidity without taking into consideration the second humidity received from the air sensor device 200 according to the identified result.

In another example, the air conditioning device 100 may identify a difference value between a first amount of fine dust (e.g., an amount of fine dust sensed by a sensor provided in the air conditioning device 100) and a second amount of fine dust (e.g., an amount of fine dust received from the air sensor device 200), and identify whether the identified difference value is greater than or equal to a threshold value. Then, the air conditioning device 100 may control an operation of the air conditioning device 100 using the first amount of fine dust without taking into consideration the second amount of fine dust received from the air sensor device 200 according to the identified result.

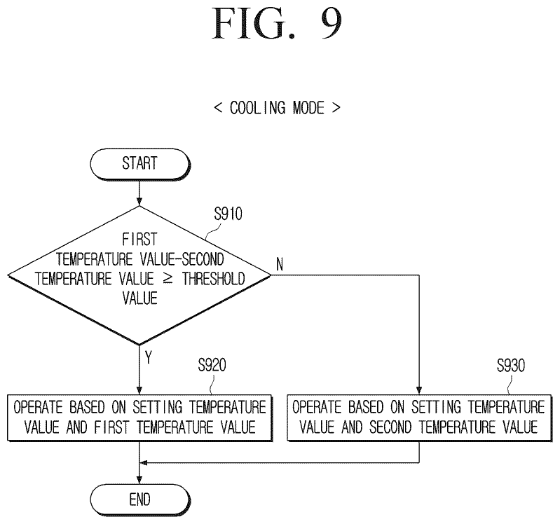

FIG. 9 is a flowchart illustrating an example method of controlling an air conditioning device in a cooling mode according to various embodiments.

A method of controlling the air conditioning device according to an embodiment may include, obtaining the first temperature value through the temperature sensor.

An operation of the compressor of the air conditioning device may be controlled based on the setting temperature value set from the air conditioning device, the first temperature value, and the second temperature value received from the air sensor device.

The controlling an operation of the compressor according to an embodiment may include identifying, based on the air conditioning device operating in the cooling mode, a difference value of subtracting the second temperature value from the first temperature value (S910), and controlling, based on the identified difference value being greater than or equal to a threshold value (S910: Y), an operation of the compressor based on the setting temperature value and the first temperature value (S920).

According to an embodiment, identifying, based on the identified difference value being greater than or equal to a threshold value (S910: Y), a relationship between a position at which air discharged from the air conditioning device reaches and a current position of the air sensor device, and transmitting information guiding of the position change of the air sensor device to the air sensor device based on the identified relationship may be further included.

Operation S910 of identifying according to an embodiment of the disclosure may include identifying, based on the identified difference value being greater than or equal to a threshold value (S910: Y), that the air sensor device is disposed at a position at which air discharged from the air conditioning device directly reaches.

According to an embodiment of the disclosure, operating, based on the difference value being less than a threshold value (S910: N), the compressor for the cooling function to be performed based on the setting temperature and the second temperature value may be included (S930).

In FIG. 9, for convenience of description, the controlling an operation of the compressor has been described as identifying whether the difference value of subtracting the second temperature value from the first temperature value is greater than or equal to the threshold value, but the above is not limited thereto.

For example, the controlling an operation of the compressor may include identifying the difference value between the first temperature value and the second temperature value, and controlling, based on the identified difference value being greater than or equal to the threshold value (e.g., identified difference value≤ (−) threshold value or, (+) threshold value≤identified difference value), an operation of the compressor based on the setting temperature value and the first temperature value.

In another example, the controlling an operation of the compressor may include controlling, based on the identified difference value being less than the threshold value (e.g., (−) threshold value≤identified difference value≤(+) threshold value), an operation of the compressor based on the setting temperature value and the first temperature value.

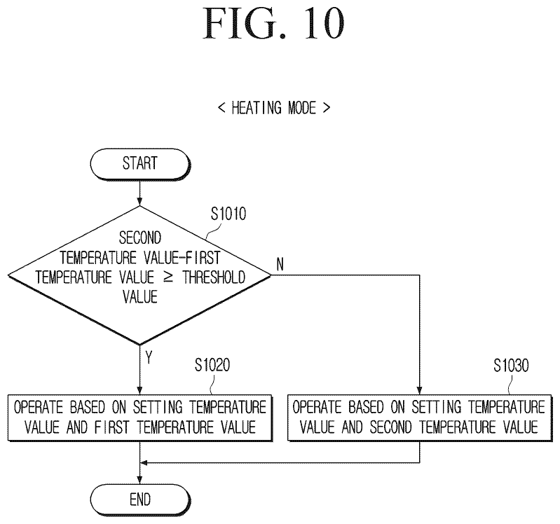

FIG. 10 is a flowchart illustrating an example method of controlling an air conditioning device in a heating mode according to various embodiments.

The controlling an operation of the compressor according to an embodiment of the disclosure may include identifying, based on the air conditioning device operating in the heating mode, a difference value of subtracting the first temperature value from the second temperature value (S1010), and controlling, based on the identified difference value being greater than or equal to the threshold value (S1010: Y), an operation of the compressor based on the setting temperature value and the first temperature value (S1020).

According to an embodiment, based on the identified difference value being greater than or equal to the threshold value (S1010: Y), identifying a relationship between a position at which air discharged from the air conditioning device reaches and a current position of the air sensor device and transmitting information guiding of the position change of the air sensor device to the air sensor device based on the identified relationship may be further included.

Operation 1010 of identifying according to an embodiment of the disclosure may include identifying, based on the identified difference value being greater than or equal to the threshold value (S1010: Y), that the air sensor device is disposed at a position at which air discharged from the air conditioning device directly reaches.

According to an embodiment of the disclosure, based on the difference value being less than the threshold value (S1010: N), operating the compressor for the cooling function to be performed based on the setting temperature and the second temperature value (S1030) may be included.

In FIG. 10, for convenience of description, the controlling an operation of the compressor has been described as identifying whether the difference value of subtracting the first temperature value from the second temperature value is greater than or equal to the threshold value, but the above is one example and is not limited thereto.

For example, the controlling an operation of the compressor may include identifying the difference value between the first temperature value and the second temperature value, and controlling, based on the identified difference value being greater than or equal to the threshold value (e.g., identified difference value≤(−) threshold value or, (+) threshold value≤identified difference value), an operation of the compressor based on the setting temperature value and the first temperature value.

In another example, the controlling an operation of the compressor may include controlling, based on the identified difference value being less than the threshold value (e.g., (−) threshold value≤identified difference value≤(+) threshold value), an operation of the compressor based on the setting temperature value and the first temperature value. According to an embodiment, the threshold value when in the cooling mode and the threshold value when in the heating mode may be the same, and may be different. For example, the threshold value when in the cooling mode may be a value relatively smaller than the threshold value when in the heating mode.

The method according to an embodiment of the disclosure may further include setting a third threshold value as a threshold value based on the first temperature value and the second temperature value being obtained at a time-point at which the first time is passed from the operation starting time-point of the air conditioning device, and setting a fourth threshold value different from the third threshold value as a threshold value based on the first temperature value and the second temperature value being obtained at a time-point at which the second time which is different from the first time is passed from the operation starting time-point of the air conditioning device.

The setting a fourth threshold value may include setting the fourth threshold value to the third threshold value to a relatively small value based on the second time being relatively longer than the first time.

The method according to an embodiment of the disclosure may further include transmitting the first temperature value to the external server, and the controlling may include controlling, based on a control signal for controlling an operation of the compressor being received from the external server according to the difference value between the first temperature value and the second temperature value, an operation of the compressor based on the control signal.

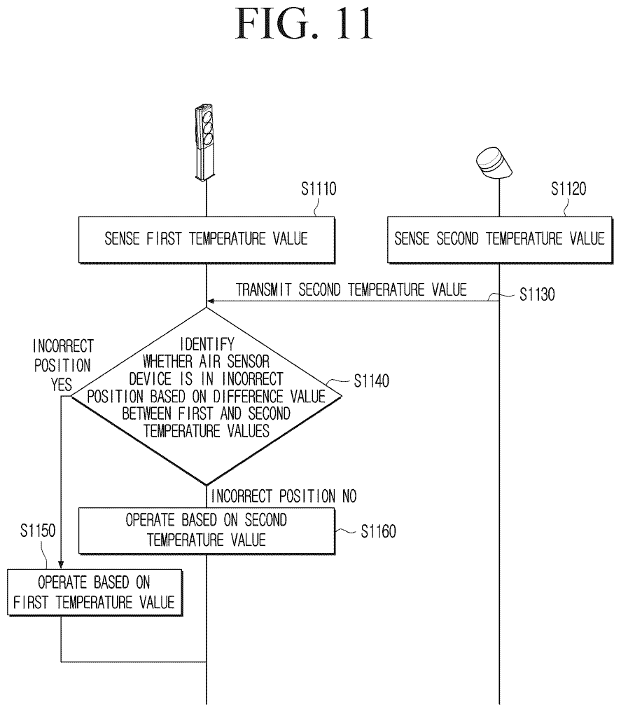

FIG. 11 is a signal flow diagram illustrating example communication between an air conditioning device and an air sensor device according to various embodiments.

Descriptions for operations that overlap with the operations described in FIG. 9 and FIG. 10 from among the steps shown in FIG. 11 may not be repeated here.

According to an embodiment of the disclosure, the air conditioning device 100 may sense the first temperature value at a time-point at which the first time is passed after the air conditioning device 100 has started operation (S1110).

According to an embodiment of the disclosure, the air sensor device 200 may sense the second temperature value at a time-point at which the first time is passed after the air conditioning device 100 has started operation (S1120), and transmit the sensed second temperature value to the air conditioning device 100 (S1130).

According to an embodiment of the disclosure, the air conditioning device 100 may identify whether the air sensor device 200 is in an incorrect position based on the difference value between the first temperature value and the second temperature value (S1140).

For example, the air conditioning device 100 may identify, based on the difference value between the first temperature value and the second temperature value being greater than or equal to the threshold value, the position of the air sensor device 200 as in the incorrect position (S1140: Y).

In another example, the air conditioning device 100 may identify, based on the difference value between the first temperature value and the second temperature value being less than the threshold value, the position of the air sensor device 200 as in a correct position (not the incorrect position) (S1140: N).

The incorrect position may refer, for example, to the air sensor device 200 being disposed at a position at which air discharged from the air conditioning device 100 directly reaches, and the temperature sensed by the air sensor device 200 is somewhat different from the indoor temperature at which the user is positioned, or somewhat different from the decreasing or increasing indoor temperature (or, a sensory temperature of the user) according to an operation of the air conditioning device 100.

If the air conditioning device 100 identifies the position of the air sensor device 200 as in the incorrect position (S1140: Y), the air conditioning device 100 may operate based on the first temperature value (S1150).

In another example, if the air conditioning device 100 identifies the position of the air sensor device 200 as in the correct position (S1140: N), the air conditioning device 100 may operate based on the second temperature value (S1160). Accordingly, the air conditioning device 100 may identify the second temperature value sensed by the air sensor device 200 as the surrounding temperature of the user (the sensory temperature of the user), and there may be an advantage of the air conditioning device 100 operating taking into consideration the surrounding temperature of the user rather than operating taking into consideration only the first temperature value sensed by the air conditioning device 100.

However, the various embodiments of the disclosure are not only applicable to the air conditioning device, but to electronic devices of all types.

The various embodiments described above may be implemented in a recordable medium which is readable by a computer or a device similar to the computer using software, hardware, or a combination thereof. In some cases, the embodiments described herein may be implemented by the processor itself. According to a software implementation, embodiments such as the procedures and functions described herein may be implemented with separate software modules. Each of the software modules may perform one or more functions and operations described herein.

Computer instructions for performing processing operations in the electronic device according to the various embodiments described above may be stored in a non-transitory computer-readable medium. The computer instructions stored in this non-transitory computer-readable medium may cause a specific device to perform a processing operation of the display device 100 according to the above-described various embodiments when executed by a processor of the specific device.

The non-transitory computer readable medium may refer to a medium that stores data semi-permanently, such as a register, a cache, a memory, or the like, and is readable by a device. Examples of the non-transitory computer readable medium may include, for example, and without limitation, a compact disc (CD), a digital versatile disc (DVD), a hard disc, a Blu-ray disc, a USB, a memory card, a ROM, and the like.

While the disclosure has been illustrated and described with reference to various embodiments thereof, it will be understood that the various embodiments are intended to be illustrative, not limiting. It will be understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

What is claimed is:

1. An air conditioning device, comprising:
a communication interface comprising circuitry;
a temperature sensor;
a compressor; and
at least one processor, comprising processing circuitry, individually and/or collectively, configured to control an operation of the compressor based on a setting temperature value set from the air conditioning device, a first temperature value obtained by the temperature sensor, and a second temperature value received from an air sensor device through the communication interface,
wherein at least one processor, individually and/or collectively, is configured to:
identify a difference value between the first temperature value and the second temperature value, and control, based on the identified difference value being greater than or equal to a threshold value, an operation of the compressor based on the setting temperature value and the first temperature value.

2. The air conditioning device of claim 1, wherein
at least one processor, individually and/or collectively, is configured to:
identify, based on the identified difference value being greater than or equal to a threshold value, a relationship between a position at which air discharged from the air conditioning device reaches and a current position of the air sensor device, and transmit information that guides of a position change of the air sensor device based on the identified relationship to the air sensor device through the communication interface.

3. The air conditioning device of claim 2, wherein
at least one processor, individually and/or collectively, is configured to:
identify, based on the identified difference value being greater than or equal to a threshold value, that the air sensor device is disposed at a position at which air discharged from the air conditioning device directly reaches, and transmit information that guides of the position change of the air sensor device to the air sensor device through the communication interface.

4. The air conditioning device of claim 1, wherein
at least one processor, individually and/or collectively, is configured to
identify, based on the air conditioning device operating in a cooling mode, whether a difference value of subtracting the second temperature value from the first temperature value is greater than or equal to a first threshold value, and
operate, based on the identified difference value being greater than or equal to the first threshold value, the compressor to perform a cooling function based on the setting temperature and the first temperature value.

5. The air conditioning device of claim 4, wherein
at least one processor, individually and/or collectively, is configured to:
operate, based on the difference value being less than the first threshold value, the compressor to perform the cooling function based on the setting temperature and the second temperature value.

6. The air conditioning device of claim 1, wherein
at least one processor, individually and/or collectively, is configured to:
identify, based on the air conditioning device operating in a heating mode, whether a difference value of subtracting the first temperature value from the second temperature value is greater than or equal to a second threshold value, and
operate, based on the identified difference value being greater than or equal to the second threshold value, the compressor to perform a heating function based on the setting temperature and the first temperature value.

7. The air conditioning device of claim 6, wherein
at least one processor, individually and/or collectively, is configured to:
operate, based on the difference value being less than the second threshold value, the compressor to perform the heating function based on the setting temperature and the second temperature value.

8. The air conditioning device of claim 1, wherein
at least one processor, individually and/or collectively, is configured to
set a third threshold value as the threshold value based on the first temperature value and the second temperature value being obtained at a point in time at which a first time is passed from an operation starting point in time of the air conditioning device, and
set a fourth threshold value different from the third threshold value as the threshold value based on the first temperature value and the second temperature value being obtained at a point in time at which a second time different from the first time is passed from an operation starting point in time of the air conditioning device.

9. The air conditioning device of claim 8, wherein
at least one processor, individually and/or collectively, is configured to:
set the fourth threshold value to the third threshold value to a value smaller than a specified threshold based on the second time being relatively longer than the first time.

10. The air conditioning device of claim 1, wherein
at least one processor, individually and/or collectively, is configured to:
transmit the first temperature value to an external server through the communication interface, and
control, based on a control signal for controlling an operation of the compressor being received from the external server according to the difference value of the first temperature value and the second temperature value, an operation of the compressor based on the control signal.

11. A method of controlling an air conditioning device comprising a temperature sensor, the method comprising:
obtaining a first temperature value through the temperature sensor; and
controlling an operation of a compressor of the air conditioning device based on a setting temperature value set from the air conditioning device, the first temperature value, and a second temperature value received from an air sensor device,
wherein the controlling comprises:
identifying a difference value between the first temperature value and the second temperature value, and controlling, based on the identified difference value being greater than or equal to a threshold value, an operation of the compressor based on the setting temperature value and the first temperature value.

12. The method of claim 11, further comprising:
identifying, based on the identified difference value being greater than or equal to the threshold value, a relationship between a position at which air discharged from the air conditioning device reaches and a current position of the air sensor device; and
transmitting information that guides of a position change of the air sensor device to the air sensor device based on the identified relationship.

13. The method of claim 12, wherein
the identifying comprises:
identifying, based on the identified difference value being greater than or equal to the threshold value, that the air sensor device is disposed at a position at which air discharged from the air conditioning device directly reaches.

14. The method of claim 11, wherein
the controlling comprises:
identifying, based on the air conditioning device operating in a cooling mode, whether a difference value of subtracting the second temperature value from the first temperature value is greater than or equal to a first threshold value; and
operating, based on the identified difference value being greater than or equal to the first threshold value, the compressor to perform a cooling function based on the setting temperature and the first temperature value.

15. The method of claim 14, wherein
the controlling comprises:
operating, based on the difference value being less than the first threshold value, the compressor to perform the cooling function based on the setting temperature and the second temperature value.

16. The method of claim 11, wherein the controlling comprises:
identifying, based on the air conditioning device operating in a heating mode, whether a difference value of subtracting the first temperature value from the second temperature value is greater than or equal to a second threshold value, and
operating, based on the identified difference value being greater than or equal to the second threshold value, the compressor to perform a heating function based on the setting temperature and the first temperature value.

17. The method of claim 16, wherein the controlling comprises:
operating, based on the difference value being less than the second threshold value, the compressor to perform the heating function based on the setting temperature and the second temperature value.

18. The method of claim 11, further comprising:
setting a third threshold value as the threshold value based on the first temperature value and the second temperature value being obtained at a point in time at which a first time is passed from an operation starting point in time of the air conditioning device, and
setting a fourth threshold value different from the third threshold value as the threshold value based on the first temperature value and the second temperature value being obtained at a point in time at which a second time different from the first time is passed from an operation starting point in time of the air conditioning device.

19. The method of claim 18, wherein the setting the fourth threshold value comprises setting the fourth threshold value to the third threshold value to a value smaller than a specified threshold based on the second time being relatively longer than the first time.

20. A non-transitory computer readable medium having instructions stored therein, which when executed by a processor cause the processor to execute method of controlling an air conditioning device comprising a temperature sensor, the method comprising:
obtaining a first temperature value through the temperature sensor; and
controlling an operation of a compressor of the air conditioning device based on a setting temperature value set from the air conditioning device, the first temperature value, and a second temperature value received from an air sensor device,
wherein the controlling comprises:
identifying a difference value between the first temperature value and the second temperature value, and controlling, based on the identified difference value being greater than or equal to a threshold value, an operation of the compressor based on the setting temperature value and the first temperature value.
